(19) United States
(12) Patent Application Publication
(10) Pub. No.: US 2012/0083684 A1
(43) Pub. Date: Apr. 5, 2012

(54) DIGITAL DISTANCE MEASURER FOR NERVE CONDUCTION STUDIES

(75) Inventors: Todd E. Kerner, Columbus, OH (US); John E. Bartsch, Hilliard, OH (US)

(73) Assignee: THE OHIO STATE UNIVERSITY, Columbus, OH (US)

(21) Appl. No.: 13/130,543

(22) PCT Filed: Nov. 20, 2009
(86) PCT No.: PCT/US09/65407

§ 371 Date: Dec. 16, 2011

Related U.S. Application Data
(62) Division of application No. 61/116,591, filed on Nov. 20, 2008.

(51) Int. Cl. A61B 5/107; A61B 6/00; A61B 5/05; A61B 5/053

(52) U.S. Cl. 600/409; 600/587; 600/547; 600/476

(57) ABSTRACT

Nerve conduction studies (NCS) are used to diagnose many types of disorders and are being employed more and more frequently. Current methods for distance measurement during a NCS are manual and often time consuming and vulnerable to considerable error. A device and system for electronically measuring distance during a NCS and automatically transferring the data is disclosed. In one embodiment, the device incorporates a contact wheel to travel across the surface of the subject's skin to measure the applicable distance.

### CROSS-REFERENCE TO RELATED APPLICATIONS

This non-provisional patent application claims the benefit of priority to U.S. Provisional patent application No. 61/116,591, filed on Nov. 20, 2008, which is hereby incorporated by reference in its entirety.

### INVENTIVE FIELD

Exemplary embodiments of the present invention are directed to a distance measuring device. More particularly, exemplary embodiments of the present invention are directed to a digital distance measuring device for use on individuals.

### BACKGROUND AND SUMMARY

A nerve conduction study (NCS) is a test commonly used to evaluate the function, especially the ability of electrical conduction, or the motor and sensory nerves of the human body. During a nerve conduction test, a nerve conduction velocity (NCV) may be measured.

Many times, nerve conduction studies are used for evaluation of paresthesias (numbness, tingling, and/or burning) and/or weakness of the arms and legs. The nerve conduction studies are used by physiatrists and neurologists to diagnose different disorders, including, but not limited to: Carpal Tunnel Syndrome, Ulnar Nerve Entrapment at Elbow, Tarsal Tunnel Syndrome, Cervical/Lumbar Radiculopathies, Gullain-Barré Syndrome, ALS (Lou Gehrig's Disease), Diabetic/other peripheral Neuropathies, and Myopathies (Duchenne, ICU, etc.).

Typically, a nerve conduction study may consist of different components. The nerve conduction study may include a motor nerve conduction study, a sensory nerve conduction study, an F-wave study, and an H-reflex study. Typically, motor nerve conduction studies are performed by electrical stimulation of a peripheral nerve and recording the output from a muscle supplied by this nerve. The time it takes for the electrical impulse to travel from the stimulation to the recording site is measured. This value is called the latency and is generally measured in milliseconds. The size of the response, called the amplitude, is also measured. Normally, motor amplitudes are measured in millivolts. By stimulating in two or more different locations along the same nerve, the NCV across different segments may be determined. Generally, calculations are performed using the distance between the different stimulating electrodes and the difference in latencies.

Usually, sensory NCS are performed by electrical stimulation of a peripheral nerve and recording from a purely-sensory portion of the nerve, such as on a finger. Like the motor studies, typically sensory latencies are also measured in milliseconds. Additionally, sensory amplitudes may be measured in microvolts. The sensory NCV is calculated based upon the latency and the distance between the stimulating and recording electrode.

During nerve conduction studies, it is typical to measure the distance between multiple areas stimulated and recorded on the body. Oftentimes, a tape measure or ruler is used to measure these distances. Sometimes, the results of the nerve conduction studies may be deficient because of the irregular, non-linear paths on the body surface.

Typically, when conducting a NCS, an individual will follow the subsequent steps: use the tape measure or similar device to record the distance, mark the location with an ink pen or similar device, stimulate the present site, manually type the distance into a computer or other operating system and repeat the entire process for each nerve tested. In an upper limb study, there are typically eight measurements captured. In a lower limb study, there are typically seven measurements captured. As a result, the typical NCS method of using a tape measure or similar device may be a time consuming, tedious, fragmented process when each measurement is repeated multiple times.

As it is realized that current devices and methods to perform a NCS may be time-consuming, tedious and fragmented, a new device and method to perform nerve conduction studies is desired. Preferably, the device and method may incorporate the stimulator with a distance measuring component that may automatically send measured values to NCS software. In some exemplary embodiments, the device and method may automatically measure distances on any skin surface without interference from conduction gel or disruption of electrical stimuli.

In some embodiments, the device may be compact and adaptable to preexisting NCS stimulators. Additionally, exemplary embodiments of the device and method may have a contact wheeled surface of size and composition that may inhibit excessive impulse dispersion. An exemplary embodiment of the device and method may increase the efficiency, time, ease and accuracy of performing a NCS. An exemplary embodiment of the present invention may provide such a distance measuring device and method.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical or equivalent features.

FIG. 1 is a depiction of a standard NCS.

FIG. 2 shows an embodiment of a contact means, an optical wheel for measuring distance, a representative output signal and exemplary equations.

FIG. 3 is a circuit diagram.

FIG. 4 shows an embodiment of a contact means, a magnet wheel for measuring distance, a representative output signal and exemplary equations.

FIG. 5 is an alternative embodiment of a representative magnet wheel apparatus.

FIG. 6 is a circuit diagram.

FIG. 7 shows an embodiment of a magnet wheel apparatus adapted to detect both forward and backward motion.

FIG. 8 shows an embodiment of a magnet wheel apparatus adapted to detect both forward and backward motion and a representative output signal for reverse motion to be compared with that of FIG. 7.

FIG. 9 is a circuit diagram.

FIG. 10 shows an alternative embodiment including a combination optical and contact means, the contact wheel arranged perpendicular to previous embodiments, and adapted to measure distance in the plane of the shaft.

FIG. 11 shows an embodiment including a means for translating the rotation of the contact wheel by 90 degrees.

FIG. 12 shows an embodiment including retractable contact wheels.

FIG. 13 shows an embodiment of a single housing unit incorporating a contact/magnet wheel and adapted to detect forward and backward motion.

### DETAILED DESCRIPTION OF EXEMPLARY EMBODIMENT(S)

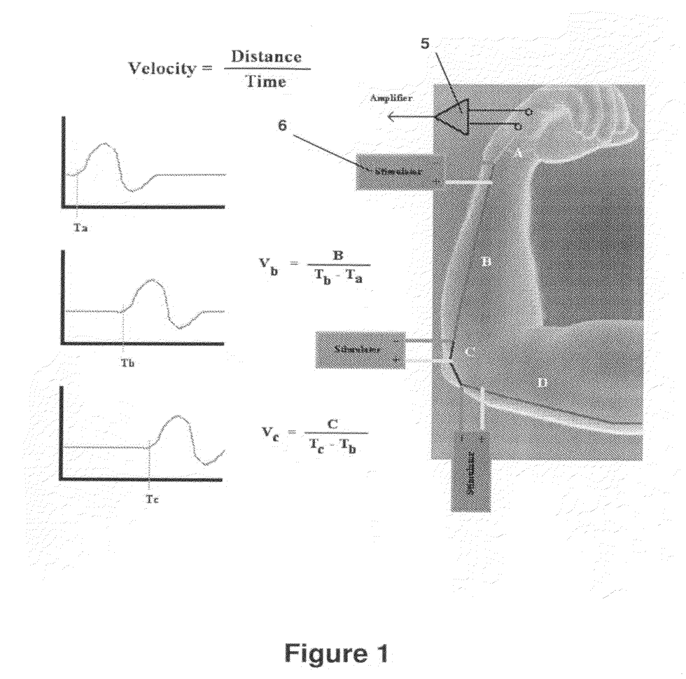

Turning to the drawings for a better understanding, FIG. 1 provides an illustration of a contemporary nerve conduction study, this one being performed on an arm nerve. A reference electrode 5 is placed at an initial position, here on the hand near the wrist of the subject. Next, a second electrode 6 (labeled as a stimulator in the Figure) is placed a certain distance from the original position, then a current is passed from the first electrode 5 to the second 6 and the time lapse is measured. Once the time lapse is measured a velocity can be determined by using the distance (A) between the initial position and the second position. It is clear that obtaining an accurate velocity value depends on the accuracy of the distance measured. This process may be repeated for the distance A several times to ensure accuracy. Next, the second electrode is moved a second distance (here denoted by B) and the excitation process is performed again. Like the first set, this second measurement may be repeated several times. This pattern is then repeated for several measured distances to complete a NCS. It is important to note here that conventional NCS distance measurements are performed using a tape measure and often a pen to mark distance on the subject. To determine the distance and the value is manually transferred to a recording device, such as a computer, which will receive the electrical stimulation data. The use of measuring devices such as a tape measure coupled with the manual data transfer expose conventional NCS measurements to significant error and needless time and effort expenditures. Clearly, a device that can minimize the error rate or time requirements is desirable. One solution is to digitally measure the distance and generate an electronic signal communicating the distance to an automated recording device.

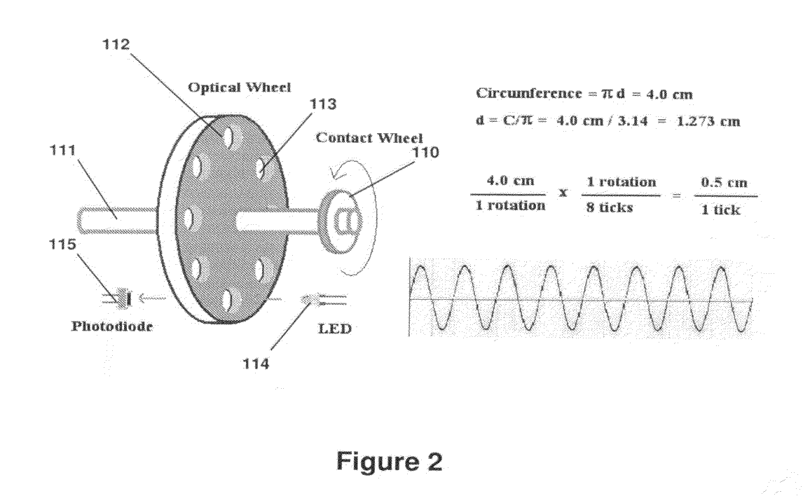

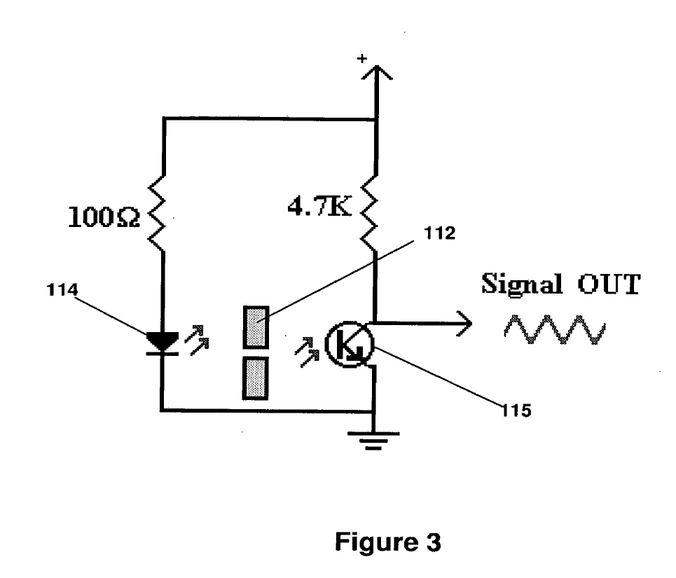

An embodiment of a NCS device incorporates a contact means 110 for measuring distance for the measurement sites. In an embodiment, the NCS digital distance measurement device may be incorporated into a housing which also includes a stimulator much like that shown in FIG. 1. This arrangement allows for more accurate distance measurement while maintaining the same number of accessories (minus a manual tape measure) as would normally be required for a NCS. FIG. 2 illustrates an embodiment wherein the contact means 110 (in this embodiment a wheel, but it is clear to one of skill that many different structures may perform this function) may be coupled to a shaft 111 which rotates an optical wheel 112. The optical wheel is termed such as it allows light to pass through at predetermined periods thus translating the distance covered by the contact wheel into radial distance. The optical wheel may have transparent sections 113 or alternatively apertures about it which allow light from an illumination source 114 (for example, an LED) to stimulate a photodetector 115. In this way each time that the photodetector is stimulated the contact means 110 has correspondingly traveled a certain distance. The photodetector then communicates via a series of electronic pulses which are interpreted by a receiver 116 (not shown) and translated into a linear distance by a recording or display device 117 (not shown). FIG. 2 also includes an example of an embodiment of the electronic signal that may be produced along with sample equations for translating the rotation into distance. FIG. 3 is an illustration of an embodiment of a circuit diagram for an optical wheel distance measurement device. The circuit diagram shows an embodiment of an optical wheel 112, illumination source 114, and photodetector 115.

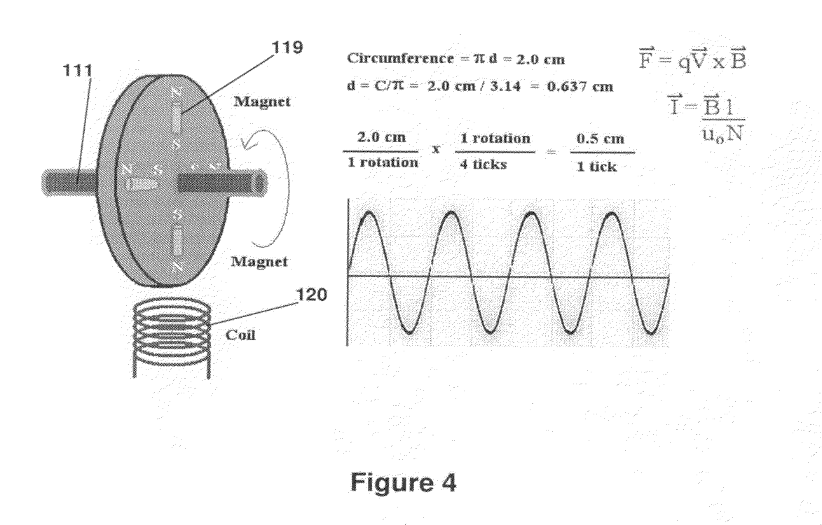

FIG. 4 illustrates an embodiment of alternative means for measuring the distance and translating the motion into an electronic signal. FIG. 4 shows an embodiment of a magnetic means 118 (here a wheel) for translating distance covered by a contact means 110 into an electronic signal. In this embodiment a magnet wheel may be coupled to a shaft 111 for translating the rotation of a contact wheel. The magnet wheel includes at least one magnet 119 about its diameter. The magnet is positioned such that it stimulates a coil 120 within the device. The coil is adapted to generate an electronic signal when a magnet comes within a certain distance. In this manner, each time that the magnet wheel rotates a certain distance (by way of the contact wheel traveling along the surface of the subject's body) the coil is stimulated by the at least one magnet on the magnet wheel and the radial motion of the contact wheel may then be translated into linear distance using the equation shown in FIG. 4 or an equivalent based on the number of magnets 119 employed in the magnet wheel.

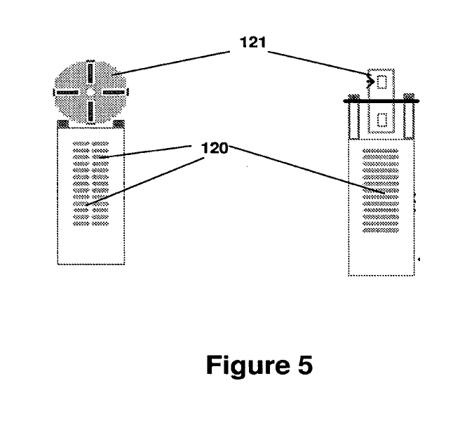

FIG. 5 shows two views of an alternative embodiment of a magnet wheel distance measuring device. In FIG. 5, the magnet wheel and the contact wheel are once again one in the same 121, thus there is no connecting shaft for translating the motion. Rather, the rotation of the contact wheel is detected by the at least one coil 120 directly. An alternative embodiment may incorporate the contact wheel, the magnet(s) and the coil(s) in a shaft that protrudes from the NCS stimulator housing.

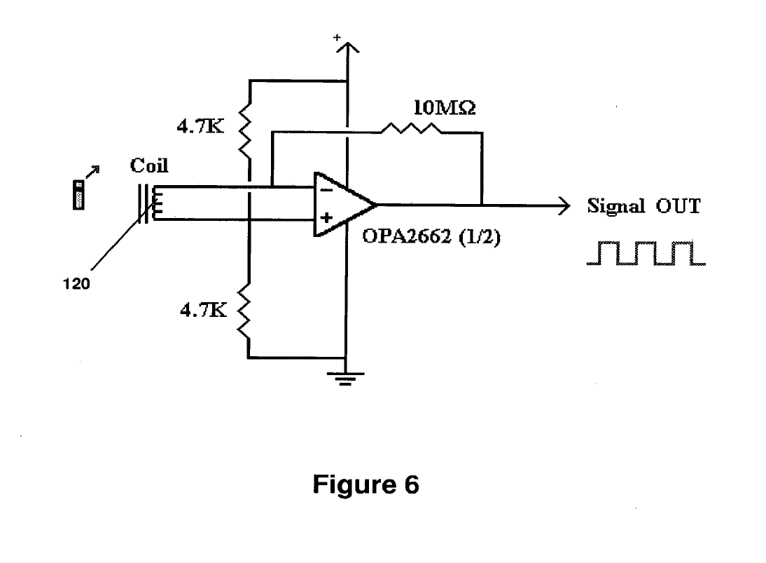

FIG. 6 is a circuit diagram for an embodiment of the NCS device. The diagram shows an alternative position for the coil(s) 120, the various electrodes and points out that the output to the recording device should be a digital signal for translating the rotation of the wheel to linear distance.

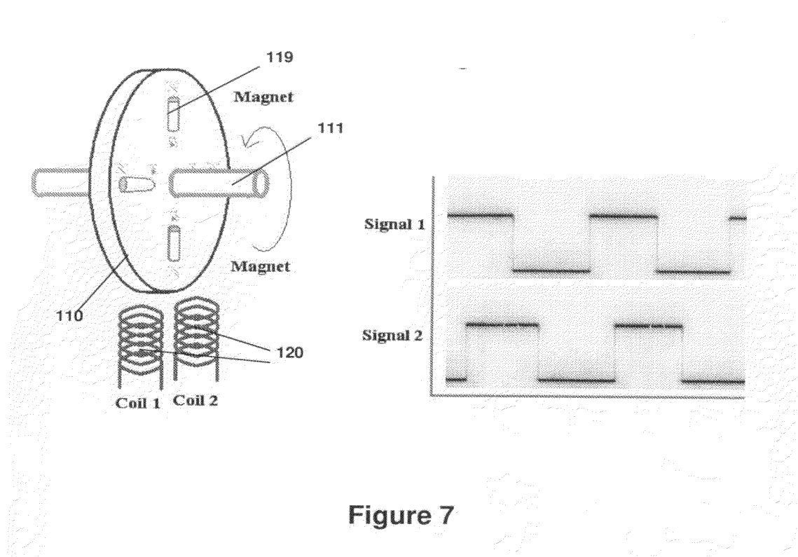

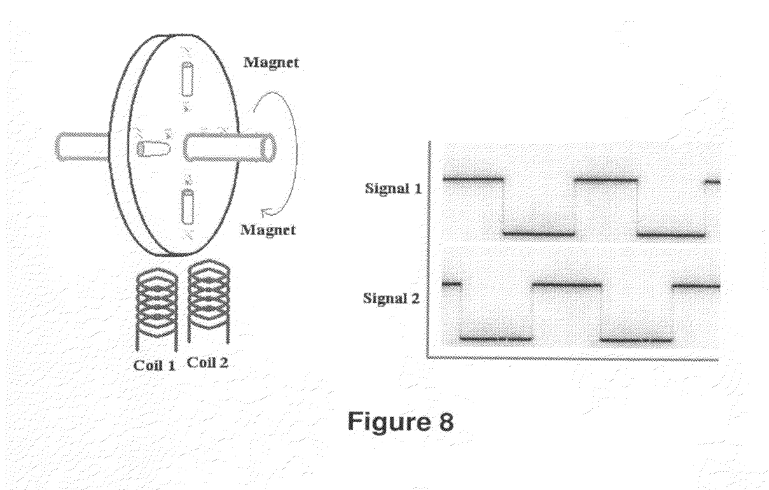

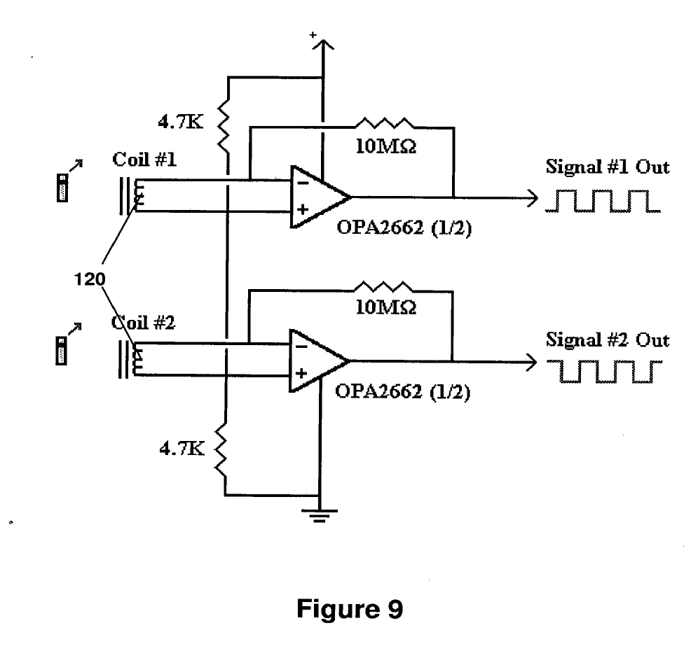

FIG. 7 is an illustration of an alternative embodiment of a NCS device. In FIG. 7, a magnet wheel 118 is again employed, however, the magnet wheel is adapted so as to interact with at least two magnetic coils 120. In this embodiment the device may then detect direction of movement and translate both forward and reverse motion. In FIG. 7, an example signal demonstrates that each magnet will pass one coil prior to the other, thus creating an offset pattern between the two coil signals. Additionally, should the contact wheel reverse course the signal would then invert allowing a receiver to detect that the contact wheel has backtracked allowing for more accurate distance measurement. Thus, if the device, and correspondingly, the contact wheel, reverse course the receiver can effectively subtract the appropriate distance from the original path's signal. FIG. 8 demonstrates this scenario and when viewed in conjunction with FIG. 7 shows that when the magnet wheel (which in this embodiment is also the contact wheel) reverses course (as shown by the two arrows in FIGS. 7 and 8), the signal pattern comparing coil 1 and coil 2 inverts. FIG. 9 is an illustration of a circuit diagram for an embodiment of an NCS device employing 2 coils 120 for digital distance measurement much like that described in FIGS. 7 and 8.

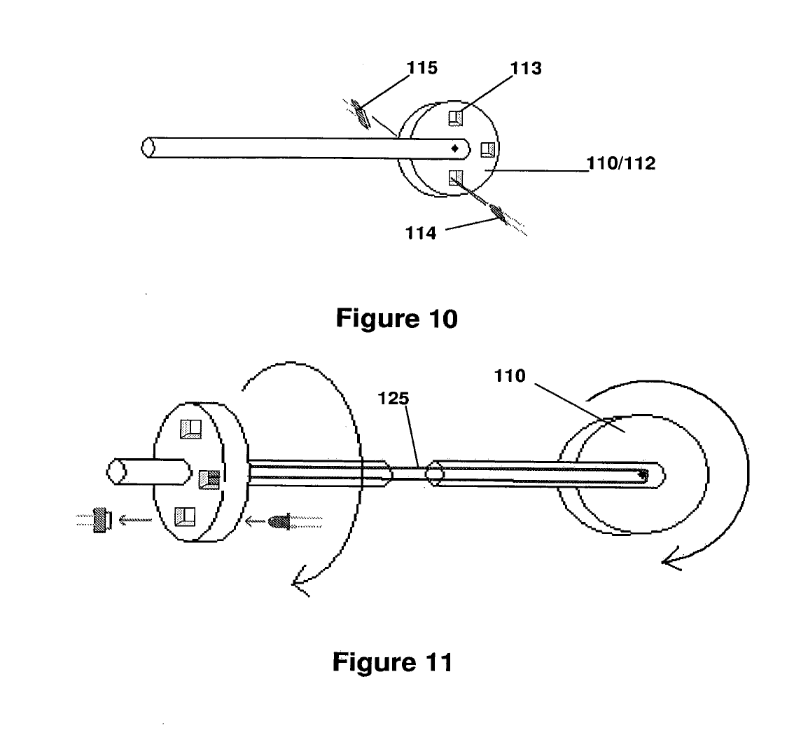

FIG. 10 shows an alternative embodiment of an NCS distance measuring device. FIG. 10 includes a feature in which the contact means 110 rotates perpendicular to that of the embodiments above. As is clear from the figure, the contact wheel may rotate in the same plane as the shaft. In FIG. 10, the contact wheel and an optical wheel have been combined. Thus rather than coupling the two wheels via a shaft or other means for communicating the rotation, the contact wheel itself may have apertures or translucent sections which allow light to pass through and excite a photodetector. Alternatively, the contact means of FIG. 10 may have reflective sections which, rather than allow light to pass through, reflect light from the illumination source toward a photodetector.

FIG. 11 shows an alternative embodiment of an NCS distance measuring device. In the embodiment of FIG. 11, the contact means 110 may be coupled to either an optical wheel or a magnet wheel (an optical wheel is shown in this embodiment merely for illustration) as described above, but in FIG. 11, the motion is translated 90 degrees by a translating means 125, thus as the contact wheel rotates in one plane across a subject's body, the translating wheel rotates in a plane approximately perpendicular to that of the contact wheel. This arrangement may allow for a smaller profile for the measuring device.

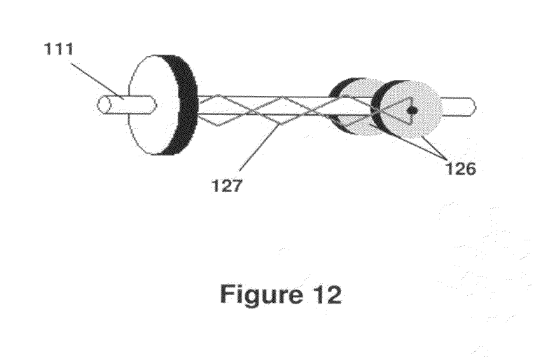

FIG. 12 shows an alternative embodiment of an NCS distance measuring device. In FIG. 12, a set of contact wheels 126 are adjacent to a probe shaft 111. The contact wheels may be attached to a means for retracting the wheels 127. Thus the wheels may be extended for measuring distance when the measuring device is passed along the subject's skin and then retracted when the device is in an appropriate position for NCS stimulation. This embodiment, like the concepts described above, may be coupled with the optical wheel, the magnet wheel or any other similar means for translating the motion of the device into a linear distance. Thus the wheel in FIG. 12 may include either of the variations described above. Alternatively, the retractable contact wheels may include a means for translating the motion themselves.

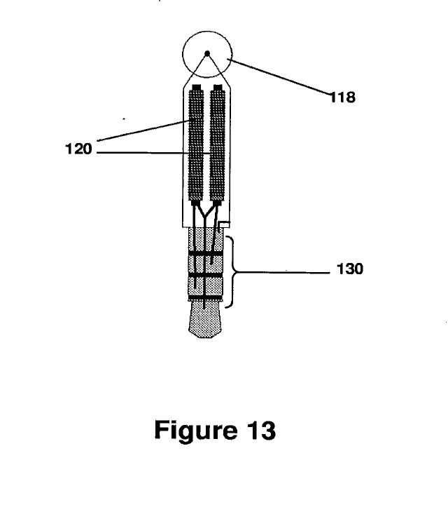

FIG. 13 shows an embodiment of a NCS distance measuring device. As is clear from the drawing, this embodiment incorporates a contact wheel which is coupled with a magnet wheel 118 for translating motion, in one housing, preferably that which houses a stimulator. The embodiment includes two coils 120 which, as described above, may translate motion and may be arranged to signal directional motion, thus the device can account for both forward and backward motion and subtract accordingly for a more accurate distance measurement. The Figure also shows an embodiment of the electrical connections for the coils and the electrode for NCS stimulation.

Any embodiment of the present invention may include any of the optional or preferred features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

What is claimed is:

1. A distance measuring device, comprising:
a housing;
a shaft adapted to rotate within the housing;
a contact means engaged to the distal end of the shaft; and
a sensing device adapted to record the movement of the contact means and generate a train of pulses.

2. The device of claim 1, wherein the sensing device is an optical sensor, comprising:
an optical wheel engaged with the shaft;
at least one photodetector; and
at least one illumination source.

3. The device of claim 1, wherein the sensing device is a magnetic sensor, comprising:
at least one magnet engaged with the contact wheel; and
at least one coil engaged with the shaft.

4. The device of claim 1, further comprising an electrode in contact with the sensing device.

5. The device of claim 1, further comprising a means for data exchange adapted to relay the train of pulses to a computer.

6. The device of claim 5, wherein the data exchange is adapted to be automated.

7. The device of claim 1, wherein the sensing device is adapted to generate pulses by illuminating a laser diode through at least one aperture in the contact means and detect the laser with a light detector.

8. The device of claim 1, further comprising a digital display adapted to display the distance measured by the device.

9. A nerve conduction testing device comprising:
a housing;
at least one electrode protruding from one end of the housing;
and electric nerve signal generator connected to the at least one electrode;
a recording device; and
a means for translating the motion of the housing to linear distance for recording by the recording device.

10. The device of claim 9, further comprising a means for data exchange adapted to relay the measured distance to the recording device.

11. The device of claim 10, wherein the means for data exchange includes a wireless transmitter capable of broadcasting a signal to a remote receiver in communication with the recording device.

12. The device of claim 11, wherein the data exchange is adapted to be automated.

13. The device of claim 9 wherein the means for translating the motion of the housing comprises:
at least one contact wheel engaged to the distal end of an electrode; and
a sensing device adapted to detect the rotation of the at least one contact wheel.

14. The device of claim 13 wherein the sensing device produces a train of pulses.

15. The device of claim 14 wherein the sensing device is an optical sensor, comprising:
an optical wheel engaged with the shaft;
at least one photodetector; and
at least one illumination source.

16. The device of claim 15, wherein the sensing device is adapted to generate pulses by detecting light generated by the at least one illumination source, through at least one aperture in the optical wheel via the at least one photodetector.

17. The device of claim 16, wherein the means for data exchange includes a wireless transmitter capable of broadcasting a signal to a remote receiver in communication with the recording device.

18. The device of claim 13, wherein the sensing device is a magnetic sensor, comprising:
at least one magnet engaged with the contact means; and
at least one coil capable of transmitting a signal to a recording device; and adapted to be stimulated by the at least one magnet.

19. The device of claim 18, wherein the means for data exchange includes a wireless transmitter capable of broadcasting a signal to a remote receiver in communication with the recording device.

20. The device of claim 19, wherein the recording device is a computer.

21. The device of claim 18, further comprising a digital display adapted to display the distance measured by the device.

22. A distance measuring device, comprising:
a housing;
at least one elongated electrode protruding from the housing;
a shaft adapted to rotate within the housing;
a contact wheel engaged to the distal end of the shaft; and
a sensing device adapted to record the movement of the contact wheel and generate a train of pulses, comprising:
at least one magnet engaged with the contact wheel;
at least one coil engaged with the shaft; and
a means for data exchange adapted to relay the train of pulses to a computer.
